(19) United States
(12) Patent Application Publication
(10) Pub. No.: US 2012/0165665 A1
(43) Pub. Date: Jun. 28, 2012

(54) METHOD FOR PROVIDING MECHANICAL INDEX MAP AND/OR PRESSURE MAP BASED ON DEPTH VALUE AND DIAGNOSTIC ULTRASOUND SYSTEM USING THE METHOD

(75) Inventors: Kurt Sandstrom, Seoul (KR); Dae Young KIM, Chuncheon-si (KR); Yoon Chang LEE, Jeongeup-si (KR); Yong Ho LEE, Seoul (KR); Tae-Yun KIM, Seoul (KR)

(73) Assignee: SAMSUNG MEDISON CO., LTD.

(21) Appl. No.: 13/092,573

(22) Filed: Apr. 22, 2011

(30) Foreign Application Priority Data
Dec. 22, 2010 (KR) 10-2010-0132633

(51) Int. Cl. A61B 8/00

(52) U.S. Cl. 600/437

(57) ABSTRACT

A diagnostic ultrasound system may visualize and display a mechanical index (MI) as a map. The diagnostic ultrasound system may include a calculating unit to calculate an MI at a depth value on an ultrasonic direction axis from an ultrasonic output unit of an ultrasonic transducer, a visualizing unit to visualize a relationship between the calculated MI and the corresponding depth value in the form of a graph to generate an MI map, and a display unit to display the MI map.

### CROSS-REFERENCE TO RELATED APPLICATION

This application claims the benefit of Korean Patent Application No. 10-2010-0132633, filed on Dec. 22, 2010, in the Korean Intellectual Property Office, the disclosure of which is incorporated herein by reference.

### BACKGROUND

1. Field of the Invention

The present invention relates to a diagnostic ultrasound system, and more particularly, to an apparatus and a method for assisting users or clinicians of a diagnostic ultrasound system in image diagnosis by providing a mechanical index (MI) map or a pressure map of a signal outputted from a pulser based on a depth value.

2. Description of the Related Art

A diagnostic ultrasound system is configured to transmit, from the surface of the body of a subject, an ultrasound wave signal toward a predetermined region inside the body, and to visualize a cross section of soft tissues or a blood flow using information of the ultrasound wave signal reflected from the tissues of the body.

The diagnostic ultrasound system has advantages of a small size, a low cost, a real-time display, and a high stability without exposing patients and users to X-ray radiation, and thus, the diagnostic ultrasound system is widely used along with other diagnostic imaging systems such as X-ray diagnosis equipment, a computerized tomography (CT) scanner, magnetic resonance imaging (MRI) equipment, nuclear medicine diagnosis equipment, and the like.

Generally, the output of the diagnostic ultrasound system including a transmission voltage, pressure, and energy is limited and determined by international guidelines, for example, a mechanical index (MI). Here, an MI is a quantitative metric of biological effects of an ultrasonic wave on the human body.

Another parameter is a thermal index (TI). Typically, the international regulatory limits for MI and TI are less than 1.9 and less than 6.0, respectively.

The diagnostic ultrasound system may diagnose an object more finely by increasing a transmission voltage of a signal outputted from a pulser, however with an increase in transmission voltage, the image quality increases and an MI also increases proportionally.

A higher MI means a larger effect of a diagnostic ultrasound system on the human body. When an MI is beyond a predetermined level, the use of a corresponding diagnostic ultrasound system is prohibited in accordance with international regulations.

Accordingly, it is possible to sufficiently increase a transmission voltage. However, when taking this problem into consideration, a transmission voltage of a diagnostic ultrasound system is finely controlled so as to maintain an MI to be less than the limit.

Conventionally, when users or clinicians of a diagnostic ultrasound system control a transmission voltage for image quality, the users or clinicians are simply provided with a reference indicated numerically without considering a distance on an ultrasonic direction axis from a pulse output unit of the diagnostic ultrasound system to a region of interest, that is, a depth of interest. In other words, the users or clinicians are not provided with a visualized reference.

### SUMMARY

An aspect of the present invention provides a method for providing a map of mechanical indices (MIs) based on a depth of interest to enable users or clinicians to easily and visually check the MIs for controlling configurable values such as a transmission output and the like, thereby improving the image quality and satisfying international MI standards, and a diagnostic ultrasound system by the method.

Another aspect of the present invention provides a method for visually providing, in addition to an MI map, a pressure map or a thermal index (TI) map, thereby improving the image quality and enabling users or clinicians to control configurable values of a diagnostic ultrasound system, and a diagnostic ultrasound system by the method.

According to an aspect of the present invention, there is provided a diagnostic ultrasound system including a calculating unit to calculate an MI at a depth value on an ultrasonic direction axis from an ultrasonic output unit of an ultrasonic transducer, a visualizing unit to visualize a relationship between the calculated MI and the corresponding depth value in the form of a graph to generate an MI map, and a display unit to display the MI map.

According to an embodiment of the present invention, the calculating unit may select a plurality of depth values on the ultrasonic direction axis from the ultrasonic output unit of the ultrasonic transducer, may calculate the MIs at each of the plurality of selected depth values, and may interpolate MIs for non-selected depth values on the ultrasonic direction axis with the calculated MIs.

The display unit may be implemented as a diagnostic monitor of at least one of the diagnostic ultrasound system and a separate user interface.

According to an embodiment of the present invention, the calculating unit may further calculate an axial pressure at a depth value on the ultrasonic direction axis from the ultrasonic output unit of the ultrasonic transducer.

In this instance, the visualizing unit may further visualize a relationship between the calculated axial pressure and the corresponding depth value in the form of a graph to generate a pressure map, and the display unit may selectively display at least one of the MI map and the pressure map.

According to another aspect of the present invention, there is provided a method for operating a diagnostic ultrasound system including calculating an MI at a depth value on an ultrasonic direction axis from an ultrasonic output unit of an ultrasonic transducer, visualizing a relationship between the calculated MI and the corresponding depth value in the form of a graph to generate an MI map, and displaying the MI map.

### EFFECT OF THE INVENTION

According to an aspect of the present invention, provided is a diagnostic ultrasound system which may provide visual information to enable users or clinicians to easily check a map of mechanical indices (MIs) based on a depth of interest, thereby satisfying international MI standards through the control of configurable values such as a transmission output and the like, and maximizing the image quality.

According to another aspect of the present invention, provided is a diagnostic ultrasound system which, in addition to an MI map, may visually provide one of a pressure map and a thermal index (TI) map, thereby satisfying international safety standards for diagnostic ultrasound equipment in various applications and improving the image quality.

According to still another aspect of the present invention, provided is a diagnostic ultrasound system which may provide information for finely controlling the use of a contrast agent or micro-bubbles in modes and applications of the diagnostic ultrasound system using a contrast agent or micro-bubbles, thereby providing convenience to users or clinicians and satisfying international safety standards for diagnostic ultrasound equipment. For example, users or clinicians may perform image diagnosis while maintaining an acoustic pressure within a predetermined range of value, which is effective for maintaining micro-bubbles.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a block diagram illustrating a diagnostic ultrasound system according to an embodiment of the present invention;

FIG. 2 is a conceptual diagram illustrating a reference axis for calculating a mechanical index (MI) or an axial pressure according to an embodiment of the present invention;

FIG. 3 is a view illustrating an example of an MI map visualized and displayed according to an embodiment of the present invention; and

FIG. 4 is a flowchart illustrating a method for operating the diagnostic ultrasound system according to an embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

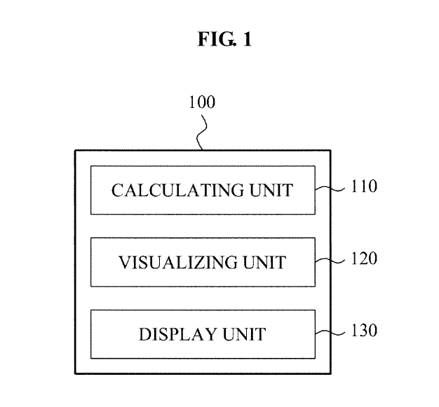

FIG. 1 is a block diagram illustrating a diagnostic ultrasound system 100 according to an embodiment of the present invention.

The diagnostic ultrasound system 100 according to an embodiment of the present invention may include a calculating unit 110, a visualizing unit 120, and a display unit 130.

The calculating unit 110 may calculate a mechanical index (MI) at a depth value on a direction axis of an ultrasonic wave emitted by the diagnostic ultrasound system 100.

The MI may be calculated by the following equation:

$$MI = \frac{{p}_{r , α} ( {z}_{MI} ) {f}_{awf}^{- 1 / 2}}{{C}_{MI}} \qquad [ Equation 1 ]$$

where C_{MI}=1 MPa·MHz^{-1/2}, and P_{r,α}(Z_{MI}) is an attenuated peak-rarefactional acoustic pressure at a depth value Z_{MI}. Also, f_{awf} is an acoustic-working frequency of the diagnostic ultrasound system 100.

In this instance, the depth value Z_{MI} is defined in international standard IEC 62369 for diagnostic imaging equipment.

According to the above standard, an MI in each mode of operation is calculated by the following equation.

(1) MI for Pulsed DTPs

$${MI}_{, pw} ( LDTP , {V}_{LDTP} ) = Voltage\_Interp \{ M1\_at \_Pii .3 \_Depth ( {V}_{LDTP} , MDTP ) \} \times \frac{\begin{matrix}Interp\_HC \_adj \_factor \_MI \\ \{ NEMA\_FcMHz ( MDTP ) , HalfCycles ( LDTP ) \}\end{matrix}}{\begin{matrix}Interp\_HC \_adj \_factor \_MI \\ \{ NEMA\_FcMHz ( MDTP ) , HalfCycles ( MDTP ) \}\end{matrix}} \times \sqrt{SysAcousticNormFactor ( Freq ( LDTP ) )} \times \sqrt{XdcrAcousticNormFactor ( Freq ( LDTP ) )} \qquad [ Equation 2 ]$$

(2) MI for CW DTPs

$$MI {,}_{cw} ( LDTP , {V}_{LDTP} ) = Voltage\_Interp \{ MI\_at \_Pii .3 \_Depth ( {V}_{LDTP} , MDTP ) \} \times \sqrt{SysAcousticNormFactor ( Freq ( LDTP ) )} \times \sqrt{XdcrAcousticNormFactor ( Freq ( LDTP ) )} \qquad [ Equation 3 ]$$

With the above equations, the calculating unit 110 may calculate MIs at depth values on an ultrasonic direction axis.

According to an embodiment of the present invention, the calculating unit 110 may not continuously calculate MIs for all depths, but may select a plurality of depth values, may calculate an MI at each of the selected depth values, and may interpolate MIs for non-selected depth values with the calculated MIs.

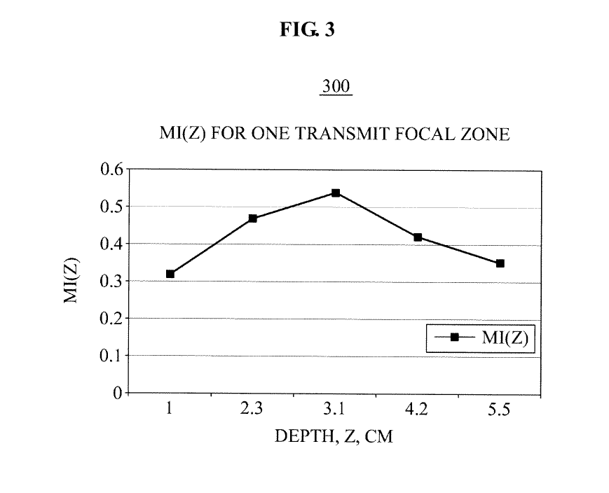

The visualizing unit 120 may visualize the calculated MIs in the form of a graph based on a depth value, which is described in further detail with reference to FIG. 3.

Through this visualizing process, a largest MI value of LDTPs may be visualized, and the largest MI value may be calculated by the following equation:

$${I}_{spta , 3 , sc} ( STOC ) = \frac{MAX}{active\_LDTPs} [ MI ( LDTP , {V}_{LDTP} ) ] \qquad [ Equation 4 ]$$

Also, MIs for depth values left out of MI calculation by the calculating unit 110 and MI visualization by the visualizing unit 120 may be calculated by the following equation, and the present embodiment is different from an embodiment using linear interpolation as described below with reference to FIG. 3.

$$MI @ depth < x > ( LDTP , {V}_{LDTP} ) = Voltage\_Interp \{ MI\_at \_depth < x > ( {V}_{LDTP} , MDTP ) \} \times \frac{\begin{matrix}Interp\_HC \_adj \_factor \_MI \\ \{ NEMA\_FcMHz ( MDTP ) , HalfCycles ( LDTP ) \}\end{matrix}}{\begin{matrix}Interp\_HC \_adj \_factor \_MI \\ \{ NEMA\_FcMHz ( MDTP ) , HalfCycles ( MDTP ) \}\end{matrix}} \times \sqrt{SysAcousticNormFactor ( Freq ( LDTP ) )} \times \sqrt{XdcrAcousticNormFactor ( Freq ( LDTP ) )} \qquad [ Equation 5 ]$$

In this instance, MIs for depth values other than predetermined depth values for which MIs have been calculated may be calculated by P_{r0.3}/\sqrt{F_{c}} for the calculated MIs. In Equation 5, ‘MI@depth<x>’ represents calculation of an MI at a depth value ‘x’ by Equation 5.

Although the above embodiments only show MI calculation and visualization, the present invention is not limited to calculation and visualization of an MI.

Another embodiment of the present invention may show calculation and visualization of an axial pressure defined in international standards, and still another embodiment of the present invention may show calculation and visualization of a TI.

The visualizing unit 120 may, in addition to an MI, selectively visualize at least one of an axial pressure and a TI.

The display unit 130 may display the result visualized in the form of a graph, that is, a map, to users or clinicians.

The display unit 130 may be implemented as a monitor of a typical diagnostic ultrasound system or other user interfaces depending on circumstances.

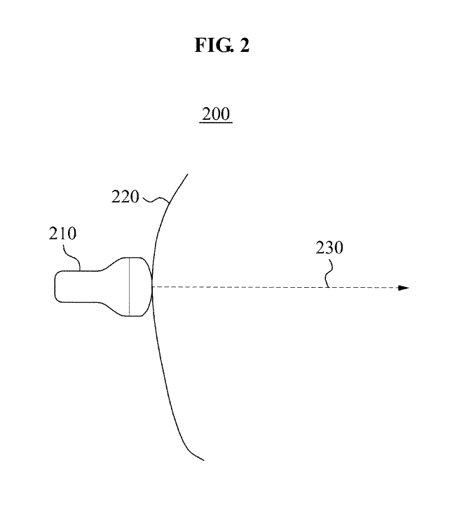

FIG. 2 is a conceptual diagram 200 illustrating a reference axis for calculating an MI or an axial pressure according to an embodiment of the present invention.

An ultrasonic direction axis from an ultrasonic transducer 210 of the diagnostic ultrasound system 100 may be an axis 230 of the depth values according to an embodiment of the present invention.

The axis 230 of the depth values may correspond to a direction of the depth values increasing within the soft tissues from a border 220 of a target to be diagnosed using the diagnostic ultrasound system 100.

FIG. 3 is a view illustrating an example of an MI map 300 visualized and displayed according to an embodiment of the present invention.

The calculating unit 110 may directly calculate an MI at each of a plurality of depth values, for example, 1, 2.3, 3.1, 4.2, and 5.5, using the above equations, and may apply a proper interpolation to other depth values, for example, linear interpolation or interpolation of Equation 5, and the visualizing unit 120 may visualize the calculated result as an MI map 300.

Another embodiment of the present invention may show one of a pressure map and an MI map, visualized using different calculation formulas from the above embodiment. However, visual representation of a map is commonly straight-forward. In this instance, calculation methods used are obvious to an ordinary person skilled in the diagnostic imaging field.

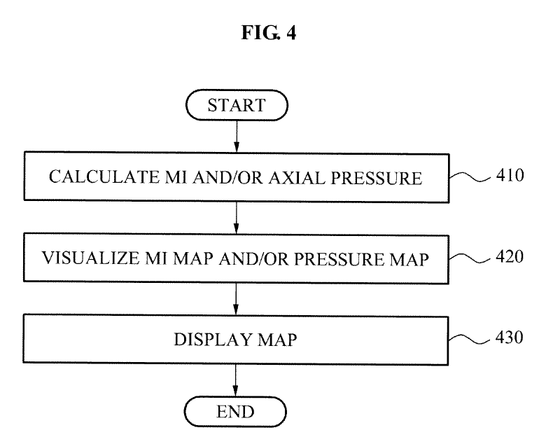

FIG. 4 is a flowchart illustrating a method for operating the diagnostic ultrasound system 100 according to an embodiment of the present invention.

In operation 410, the calculating unit 110 of the diagnostic ultrasound system 100 may calculate at least one of an MI and an axial pressure at a depth value on an ultrasonic direction axis.

At least one of MIs for a plurality of selective depth values and axial pressure for the plurality of selective depth values may be first calculated, and a proper interpolation may be applied to the other depth values, as described above with reference to FIGS. 1 and 2.

In operation 420, the visualizing unit 120 may visualize at least one of the calculated result and interpolated result, in the form of a graph, and an example of an MI map is described above with reference to FIG. 3.

In operation 430, the display unit 130 may display at least one of an MI map and a pressure map, or, in another embodiment, may display a TI map.

This displayed map may be used for users or clinicians to control various configurable values related to an ultrasonic output so that the diagnostic ultrasound system 100 may perform a more accurate imaging operation and thus, the displayed map may assist the users or clinicians or the diagnostic ultrasound system 100 to recognize the control limits of the configurable values so as to prevent the configurable values from deviating from international safety standards.

Accordingly, the embodiments of the present invention may provide visual information to enable users or clinicians to easily check a map of MIs based on a depth of interest, thereby satisfying international MI standards through the control of configurable values such as a transmission output and the like, and maximizing the image quality.

Also, the embodiments of the present invention may, in addition to an MI map, visually provide one of a pressure map and a TI map, thereby satisfying international safety standards for diagnosis equipment in various applications, and improving the image quality.

Also, the embodiments of the present invention may provide information for finely controlling the use of at least one of a contrast agent and micro-bubbles in modes, and for finely controlling applications of a diagnostic ultrasound system using at least one of a contrast agent and micro-bubbles, thereby providing convenience to users or clinicians and satisfying international safety standards for diagnostic ultrasound equipment. For example, users or clinicians may perform image diagnosis while maintaining an acoustic pressure within a predetermined range of value, which is effective for maintaining micro-bubbles.

The above-described exemplary embodiments of the present invention may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described exemplary embodiments of the present invention, or vice versa.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A diagnostic ultrasound system comprising:
a calculating unit to calculate a mechanical index (MI) at a depth value on an ultrasonic direction axis from an ultrasonic output unit of an ultrasonic transducer;
a visualizing unit to visualize a relationship between the calculated MI and the corresponding depth value in the form of a graph to generate an MI map; and
a display unit to display the MI map.

2. The system of claim 1, wherein the calculating unit selects a plurality of depth values on the ultrasonic direction axis from the ultrasonic output unit of the ultrasonic transducer, calculates the MIs at each of the plurality of selected depth values, and interpolates MIs for non-selected depth values on the ultrasonic direction axis with the calculated MIs.

3. The system of claim 1, wherein the display unit is implemented as a diagnostic monitor of at least one of the diagnostic ultrasound system and a separate user interface.

4. The system of claim 1, wherein the calculating unit further calculates an axial pressure at a depth value on the ultrasonic direction axis from the ultrasonic output unit of the ultrasonic transducer.

5. The system of claim 4, wherein the visualizing unit further visualizes a relationship between the calculated axial pressure value and the corresponding depth value in the form of a graph to generate a pressure map, and
the display unit selectively displays at least one of the MI map and the pressure map.

6. A method for operating a diagnostic ultrasound system, the method comprising:
calculating a mechanical index (MI) at a depth value on an ultrasonic direction axis from an ultrasonic output unit of an ultrasonic transducer;
visualizing a relationship between the calculated MI and the corresponding depth value in the form of a graph to generate an MI map; and
displaying the MI map.

7. The method of claim 6, wherein the calculating comprises:
selecting a plurality of depth values on the ultrasonic direction axis from the ultrasonic output unit of the ultrasonic transducer;
calculating an MI at each of the plurality of selected depth values; and
interpolating MIs for non-selected depth values on the ultrasonic direction axis with the MI calculated at each of the plurality of selected depth values.

8. The method of claim 6, wherein the displaying is implemented as a diagnostic monitor of the diagnostic ultrasound system or a separate user interface.

9. The method of claim 6, wherein the calculating comprises further calculating an axial pressure at a depth value on the ultrasonic direction axis from the ultrasonic output unit of the ultrasonic transducer.

10. The method of claim 9, wherein the visualizing comprises further visualizing a relationship between the calculated axial pressure value and the corresponding depth value in the form of a graph to generate a pressure map, and
the displaying selectively displays at least one of the MI map and the pressure map.
